(19) United States
(12) Patent Application Publication
(10) Pub. No.: US 2025/0332081 A1
(43) Pub. Date: Oct. 30, 2025

(54) A Composition Against Osmotic Stress

(71) Applicant: ELC Management LLC, Melville, NY (US)

(72) Inventors: Yulan QU, Pudong District, Shanghai (CN); Nan HUANG, Pudong District, Shanghai (CN)

(21) Appl. No.: 18/832,510

(22) PCT Filed: Jan. 26, 2022
(86) PCT No.: PCT/CN2022/073938

§ 371 Date: Jul. 24, 2024

(51) Int. Cl. A61K 8/44; A61K 8/9789; A61Q 5/00; A61Q 19/00; A61Q 19/08

(52) U.S. Cl. CPC A61K 8/44; A61K 8/9789; A61Q 5/00; A61Q 19/007; A61Q 19/08

(57) ABSTRACT

The subject matter of the present application is a composition, especially cosmetic and/or dermatological composition comprising a combination of a roselle extract and a betaine compound, as well as a non-therapeutic cosmetic treatment process of the skin to fight against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

### TECHNICAL FIELD

The present invention relates to the field of cosmetic or dermatologic compositions. In particular, the present invention is directed to new cosmetic and/or dermatological composition for topical application, comprising a roselle extract and a betaine compound.

The present invention also relates to the cosmetic use of said composition, and in particularly for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

### BACKGROUND ART

The skin is one of the most important organs of the human body. It exists on the outermost part of the body as a barrier to protect the human body from the adverse effects of the external environment. The keratinocytes at different stages of differentiation constitute the five-layer structure of the skin's epidermis, e.g., stratum corneum, stratum lucidum, stratum granulosum, stratum spinosum, and stratum basale, from the outside to the inside. The epidermis composed of keratinocytes serves as a barrier function to inhibit the transmission of foreign bodies and prevent the loss of transepidermal water. However, with the development of modern society, skin cells are facing continuous threat of osmotic press due to continuous exposure to ultraviolet rays, air pollution, dry environment, chemical substances, etc. As an agent for protecting skin cells subjected to such stress, FR2877843 discloses the use of betaine in the field of cosmetics to combat skin aging and damage.

Further, as a cosmetic composition for protecting the skin from different types of stress, US20100166814 provides a composition comprising a combination of at least two osmolytes selected from the group comprising taurine or a derivative thereof, inositol, betaine and trehalose.

Recently, it is reported that the use of betaine in combination with various plant extracts can not only achieve moisturizing, but also achieve the additional advantageous effects brought by the various plant extracts. For example, CN104523490 reports that betaine is used in combination with moisturizing plant extracts and whitening plant extracts, thereby providing an exfoliating composition with whitening and moisturizing effects. Moreover, US20160287658 reports that the combination of betaine and bamboo sap can significantly increase the water content in the stratum corneum. CN105411900 teaches that the combination of betaine and the Leontopodium alpinum extract can enhance the effect of Leontopodium alpinum.

Hibiscus sabdariffa L. is a plant of the genus Hibiscus of the family Miancaceae, and has value as a food raw material, natural pigment, and medical use. It is used in an anti-allergic composition in CN108653090 due to its antioxidant properties and tyrosinase inhibitory effect. In addition, as described in CN104523490, Hibiscus sabdariffa flower extract is mentioned as a whitening plant extract.

To the knowledge of the inventors, the synergistic protective property against osmotic stress of the combination of the roselle extract and the betaine compound has never been disclosed.

### SUMMARY OF THE INVENTION

The inventors have intensively studied to solve the problem that is to provide a combination which can produce a synergistic protective property against osmotic stress.

As a result, the inventors have found that the synergistic protective property against osmotic stress can be produced by combining the betaine compound and the roselle extract in a specific manner.

In other words, in a first aspect, the present application provides a composition, especially cosmetic and/or dermatological composition for topical application, comprising a roselle extract and at least a betaine compound selected from the group consisting of betaine, its salt and its analogs.

In an embodiment of the present application, the betaine compound is glycine betaine.

In another embodiment of the present application, the roselle extract is from the calyx or fruit of the roselle.

In a further embodiment of the present application, the roselle extract is extracted by a water-based extraction process.

In a further embodiment of the present application, the betaine compound and the roselle extract are present in a weight ratio from 3:1 to 3:10, preferably from 3:1 to 6:10 and more preferably from 9:5 to 9:10.

In a further embodiment of the present application, the betaine compound is present in an amount from 0.01% to 0.2% by weight, and preferably from 0.03% to 0.1% by weight, relative to the total weight of the composition.

In a further embodiment of the present application, the roselle extract is present in an amount from 0.01% to 0.1% by weight, preferably from 0.02% to 0.1% by weight and more preferably from 0.05% to 0.1% by weight, relative to the total weight of the composition.

In a second aspect, the present application provides non-therapeutic cosmetic treatment process, comprising the topical application to keratin materials such as the skin of the above-mentioned composition.

In an embodiment of the present application, the above-mentioned process is for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

In another embodiment of the present application, the stress factor is UV radiation, chemical and/or osmotic stress.

In a third aspect, the present application provides cosmetic use of the above-mentioned composition, for caring for, protecting and/or making up the skin of the body or of the face, or for caring for the hair.

In an embodiment of the present application, the above-mentioned use is for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

In another embodiment of the present application, the stress factor is UV radiation, chemical and/or osmotic stress.

The above-mentioned composition can provide a synergistic protective property against osmotic stress by comprising the combination of the betaine compound and the roselle extract in an appropriate ratio and concentration range, thereby reducing stress-induced cell damage and fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

### DETAILED DESCRIPTION OF THE INVENTION

### I. Definitions

In that which follows and unless otherwise indicated, the limits of a range of values are included within this range, in particular in the expressions “between . . . and . . . ” and “from . . . to . . . ”.

Unless defined otherwise, all technical and scientific terms used herein have the same meaning as commonly understood by one of ordinary skill in the art to which the present invention pertains. When the definition of a term in the present description conflicts with the meaning as commonly understood by those skilled in the art the present invention belongs to, the definition described herein shall apply.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

The term “skin cells” used herein represents cells that make up skin including but not limited to keratinocyes, melanocytes, fibroblasts, T-cells, and so on.

The term “UV radiation” used herein represents ultraviolet radiation in the UVA and UVB wavelength ranges.

The term “stress” used herein represents stress conditions to which skin cells may be exposed such as ultraviolet rays, air pollution, dry environment, chemical substances, or osmotic stress. Here, the osmotic stress means a physiologic dysfunction caused by a sudden change in the solute concentration around a cell, which causes a rapid change in the movement of water across its cell membrane. Under hypertonic conditions—conditions of high concentrations of either salts, substrates or any solute in the supernatant—water is drawn out of the cells through osmosis.

The term “synergistic protective property” as used herein represents that the protective effect produced by the combined use of the two active ingredients, for example, at least one betaine compound (for example, trimethylglycine) and roselle extract (for example, the extract from Hibiscus sabdariffa L.) of the present invention, is greater than the sum of the protective effects when the two active ingredients are used alone at the same dose.

Unless otherwise specified, all numerical values expressing amount of ingredients and the like which are used herein are to be understood as being modified by the term “about”.

Unless otherwise specified, all percentages mentioned herein are percentages by weight, and all documents mentioned herein are incorporated by reference in their entirety.

### II. Roselle Extract

The composition of the invention contains roselle extract from Hibiscus sabdariffa L., which (also referred as Rosella or Roselle) is a plant of the genus Hibiscus of the family Miancaceae, and has value as a food raw material, natural pigment, and medical use. The extract from Hibiscus sabdariffa L. provide improved skin texture, feel, hydration, moisturization, and appearance of fine lines and wrinkles, the withering, the thinning, the dryness, the looseness, the dull appearance, or the heterogeneous surface, of the skin or other undesirable skin conditions.

The roselle extract used herein may be obtained from the plant parts selected from the group consisting of flowers, seeds, leaves, fruits, calyx, or roots of the Hibiscus sabdariffa L. Preferably, the roselle extract used herein may be obtained from the plant parts selected from the group consisting of flowers, calyx, fruits of Hibiscus sabdariffa L. More preferably, the roselle extract used herein may be obtained from the fruits of Hibiscus sabdariffa L.

The roselle extract used herein may be an aqueous or aqueous/alcoholic extract of the plant parts. Preferably, the roselle extract used herein may be extracted by an aqueous extraction process. Preferably, the roselle extract used herein may be purchased from Lucas Meyer Cosmetics Company under the tradename Hibiscus Sabdariffa Extract, which is obtained by aqueous extraction in the presence of enzyme and pH adjustment followed by lyophilization. The Hibiscus Sabdariffa Extract is also commercially available as CAS No.84775-96-2 or EC No. 283-920-7.

The roselle extract used herein may be present in an amount from 0.01% to 0.1% by weight, preferably from 0.02% to 0.1% by weight and more preferably from 0.05% to 0.1% by weight, relative to the total weight of the composition. In one embodiment, the roselle extract contained in the composition may be present in an amount of 0.01%, 0.02%, 0.03%, 0.04%, 0.05%, 0.06%, 0.07%, 0.08%, 0.09%, 0.1% by weight, relative to the total weight of the composition.

While not wishing to be bound by theory, it is believed that if the amount of the roselle extract is less than 0.01% by weight relative to the total weight of the composition, the roselle extract could not exhibit a synergistic effect with the following betaine compound because the amount is too low. However, if the amount is higher than 0.1% by weight relative to the total weight of the composition, it may adversely affect the skin cells due to the cytotoxicity of high concentration.

### III. Betaine Compound

The composition of the invention contains at least a betaine compound selected from the group consisting of betaine, its salt and its analog. The term “betaine” used herein represents the general name for the intramolecular salt formed of a quaternary ammonium as a cation and an anion of an acid, particularly a carboxylic acid, in one molecule.

The betaine compound suitable for use in the composition of the present invention include those known to be useful in cosmetic or dermatological compositions.

In one embodiment, the betaine used herein may be selected from the group consisting of histidine betaine, glycine betaine, proline betaine, phenylalanine betaine, tyrosine betaine, tryptophan betaine, leucine betaine, isoleucine betaine, valine betaine, betaine glutamate, alanine betaine, betaine aspartate, lysine betaine, and arginine betaine depending on the amino acid involved.

In another embodiment, the betaine used herein may be selected from the group consisting of alkyl betaines, alkyl amidobetaines, alkyl sulfobetaines, and imidazolinium betaines, in particular, alkyl N-betaines, alkyl N-sulfobetaines, acyl N-betaines, alkyl N-substituted aminopropionic acid, alkyl imidazolinium betaines, etc.

Further, the betaine compound suitable for use in the composition of the present invention include the salt and the analog of the betaine as listed above.

In one embodiment, as the salt of betaine, an organic acid salt, an inorganic acid salt or a base salt of the above-mentioned betaine may be used. In particular, betaine hydrochloride, betaine hydrobromide, betaine hydroiodide, betaine chloroaurate, betaine hydrazide hydrochloride, etc. may be used.

In a preferred embodiment, the betaine used herein may be glycine betaine, which is also called as trimethylglycine having the following structure.

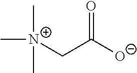

In a preferred embodiment, the betaine used herein may be glycine betaine which is commercially available as CAS No. 107-43-7.

Moreover, the term “analog” used herein represents any compound having similar properties with betaine, in particular, trimethylglycine.

The betaine compound used herein may be present in an amount from 0.01% to 0.2% by weight, and preferably from 0.03% to 0.1% by weight, relative to the total weight of the composition. In one embodiment, the betaine compound contained in the composition of the invention may be present in an amount of 0.01%, 0.02%, 0.03%, 0.04%, 0.05%, 0.06%, 0.07%, 0.08%, 0.09%, 0.1% or 0.2% by weight, relative to the total weight of the composition.

While not wishing to be bound by theory, it is believed that if the amount of the betaine compound is less than 0.01% by weight relative to the total weight of the composition, the betaine compound could not exhibit a synergistic effect with the roselle extract because the amount is too low. However, if the amount is higher than 0.1% by weight relative to the total weight of the composition, it will not increase the synergistic effect, it is thus not preferable from the view of cost.

The composition of the invention contains a roselle extract and at least a betaine compound as defined above. In one embodiment, the betaine compound and the roselle extract are in a weight ratio from 3:1 to 3:10, preferably from 3:1 to 6:10 and more preferably from 9:5 to 9:10.

### IV. Other Ingredients

The composition of the invention may be in the solid, semi-solid, or liquid form, and may be in the solution, emulsion, suspension, or anhydrous form. If in the solution or suspension form, the composition may contain from about 1-99.9%, preferably from about 5-95%, more preferably from about 10-90% water. If in the emulsion form, the composition may contain from about 1-99%, preferably from about 5-90%, more preferably from about 10-85% water and from about 1-99%, preferably from about 5-90%, more preferably from about 5-75% of oil. If in the anhydrous form, the composition may contain from about 10-99% oil and 10-99% solidifying agents.

As long as the synergistic protective effect of the composition of the present invention is not affected, the composition of the present invention may contain other ingredients, including but not limited to those described herein.

### A. Humectants

The composition of the invention may contain one or more humectants. If present, they may range from about 0.1 to 75%, preferably from about 0.5 to 70%, more preferably from about 0.5 to 40%. Examples of suitable humectants include glycols, sugars, and the like. Suitable glycols are in monomeric or polymeric form and include polyethylene and polypropylene glycols such as PEG 4-10, which are polyethylene glycols having from 4 to 10 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol.

### B. Botanical Extracts

The composition of the invention may contain one or more additional botanical extracts other than the roselle extract. If present suggested ranges are from about 0.0001 to 20%, preferably from about 0.0005 to 15%, more preferably from about 0.001 to 10%. Suitable botanical extracts include extracts from plants (herbs, roots, flowers, fruits, seeds) such as flowers, fruits, vegetables, and so on, including yeast ferment extract, Padina pavonica extract, Thermus thermophilis ferment extract, Camelina sativa seed oil, Boswellia serrata extract, olive extract, Acacia dealbata extract, Acer saccharinum (sugar maple), Acidopholus, Acorus, Aesculus, Agaricus, Agave, Agrimonia, algae, aloe, citrus, Brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza glabra, Salix nigra, Macrocycstis pyrifera, Pyrus malus, Saxifraga sarmentosa, Vitis vinifera, Morus nigra, Scutellaria baicalensis, Anthemis nobilis, Salvia sclarea, Rosmarinus officianalis, Citrus limonum, Panax ginseng, Siegesbeckia orientalis, Fructus mume, Ascophyllum nodosum, Glycine soja extract, Beta vulgaris, Haberlea rhodopensis, Polygonum cuspidatum, Citrus aurantium dulcis, Vitis vinifera, Selaginella tamariscina, Humulus lupulus, Citrus reticulata Peel, Punica granatum, Asparagopsis, Curcuma longa, Menyanthes trifoliata, Helianthus annuus, Hordeum vulgare, Cucumis sativus, Evernia prunastri, Evernia furfuracea, Kola acuminata, and mixtures thereof.

### C. Surfactants

The composition of the invention may contain one or more surfactants, especially if in the emulsion form. However, such surfactants may be used if the compositions are solutions, suspensions, or anhydrous also. If present, the surfactant may range from about 0.001 to 30%, preferably from about 0.005 to 25%, more preferably from about 0.1 to 20% by weight of the total composition. Suitable surfactants may be silicone or organic, nonionic, anionic, amphoteric or zwitterionic.

### 1. Organic Nonionic Surfactants

The composition of the invention may contain one or more nonionic organic surfactants. Suitable nonionic surfactants include alkoxylated alcohols or ethers, formed by the reaction of an alcohol with an alkylene oxide, usually ethylene or propylene oxide. Suitable alcohols include mono-, di-, or polyhydric short chain (C₁₋₆) alcohols; aromatic or aliphatic saturated or unsaturated fatty (C₁₂₋₄₀) alcohols, of cholesterol; and so on.

Cholesterol is suitable, or an aromatic or aliphatic saturated or unsaturated fatty alcohol which may have from 6 to 40, preferably from about 10 to 30, more preferably from about 12 to 22 carbon atoms. Examples include oleyl alcohol, cetearyl alcohol, cetyl alcohol, stearyl alcohol, isostearyl alcohol, behenyl alcohol, and the like. Examples of such ingredients include Oleth 2-100; Steareth 2-100; Beheneth 5-30; Ceteareth 2-100; Ceteth 2-100; Choleth 2-100 wherein the number range means the number of repeating ethylene oxide units, e.g. Ceteth 2-100 means Ceteth where the number of repeating ethylene oxide units ranges from 2 to 100. Derivatives of alkoxylated alcohols are also suitable, such as phosphoric acid esters thereof.

Some preferred organic nonionic surfactants include Oleth-3, Oleth-5, Oleth-3 phosphate, Choleth-24; Ceteth-24; and so on.

Also suitable are alkoxylated alcohols formed with mono-, di-, or polyhydric short chain alcohols, for example those having from about 1 to 6 carbon atoms. Examples include glucose, glycerin, or alkylated derivatives thereof. Examples include glycereth 2-100; gluceth 2-100; methyl gluceth 2-100 and so on. More preferred are methyl gluceth-20; glycereth-26 and the like.

Other types of alkoxylated alcohols are suitable surfactants, including ethylene oxide polymers having varying numbers of repeating EO groups, generally referred to as PEG 12 to 200. More preferred are PEG-75, which is may be purchased from Dow Chemical under the trade name Carbowax PEG-3350.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C₆₋₃₀, preferably C₁₂₋₂₂ fatty acids. Examples of such ingredients include Polysorbates 20-85, sorbitan oleate, sorbitan sesquioleate, sorbitan palmitate, sorbitan sesquiisostearate, sorbitan stearate, and so on.

### 2. Silicone or Silane Surfactants

Also suitable are various types of silicone or silane-based surfactants. Examples include organosiloxanes substituted with ethylene oxide or propylene oxide groups such as PEG dimethicones which are dimethicones substituted with polyethylene glycols including those having the INCI names PEG-1 dimethicone; PEG-4 dimethicone; PEG-8 dimethicone; PEG-12 dimethicone; PEG-20 dimethicone; and so on.

Also suitable are silanes substituted with ethoxy groups or propoxy groups or both, such as various types of PEG methyl ether silanes such as bis-PEG-18 methyl ether dimethyl silane; and so on.

Further examples of silicone based surfactants include those having the generic names dimethicone copolyol; cetyl dimethicone copolyol; and so on.

### D. Biological Materials

The composition of the invention may contain various types of biological materials such as those derived from cells, fermented materials, and so on. If present such materials may range from about 0.001 to 30%, preferably from about 0.005 to 25%, more preferably from about 0.01 to 20%. Examples include fragments of cellular RNA or DNA, or probiotic microorganisms. Particularly preferred are RNA fragments.

### E. Thickeners

Suitable thickeners may be incorporated into the composition of the invention. If present, suggested ranges are from about 0.01 to 30%, preferably from about 0.1 to 20%, more preferably from about 0.5 to 15% by weight of the total composition.

Examples of thickeners include animal, vegetable, mineral, silicone, or synthetic waxes which may have melting points ranging from about 30 to 150° C. including but not limited to Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

Also suitable as thickening agents are silicas, silicates, silica silylate, and alkali metal or alkaline earth metal derivatives thereof. These silicas and silicates are generally found in the particulate form and include silica, silica silylate, magnesium aluminum silicate, and the like.

Silicone elastomers may also be used as thickening agents. Such elastomers include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or a network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C1-22 alkyl acrylate, a C1-22 alky methacrylate, and mixtures thereof are suitable. Acrylic polymer solutions include those sold by Seppic, Inc., under the tradename Sepigel® or those sold under the tradename Aristoflex®.

Also suitable are acrylic polymeric thickeners that are copolymer of A, B, and C monomers wherein A and B are as defined above, and C has the general formula:

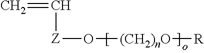

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specificially a vinyl carboxylic acid such as acrylic acid, methacrylic acid or mixtures thereof, and where the allyl ether unit containing a fatty chain corresponds to the monomer of formula:

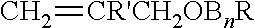

wherein R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C₁₈) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C10-30 alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

Also suitable as thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CTFA names polyglycerin-20, polyglycerin-40, and the like.

### F. Oils

In the case that the compositions of the invention are in emulsion form, the composition will contain an oil phase. Oily ingredients are desirable for the skin moisturizing and protective properties. Suitable oils include silicones, esters, vegetable oils, synthetic oils, including but not limited to those set forth herein. The oils may be volatile or nonvolatile, and are preferably in the form of a pourable liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

### 1. Volatile Oils

Suitable volatile oils generally have a viscosity ranging from about 0.5 to 5 centistokes 25° C. and include linear silicones, cyclic silicones, paraffinic hydrocarbons, or mixtures thereof.

### (a). Volatile Silicones

Cyclic silicones are one type of volatile silicone that may be used in the composition. Such silicones have the general formula:

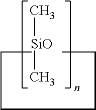

wherein n=3-6, preferably 4, 5, or 6.

Also suitable are linear volatile silicones, for example, those having the general formula:

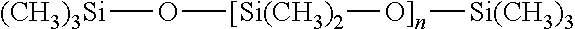

wherein n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

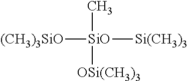

Methyl trimethicone may be purchased from Shin-Etsu Silicones under the tradename TMF-1.5, having a viscosity of 1.5 centistokes at 25° C.

### (b). Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference.

Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

### 2. Non-Volatile Oils

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

### (a). Esters

Suitable esters are mono-, di-, and triesters. The composition may comprise one or more esters selected from the group, or mixtures thereof.

### (i) Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

### (ii). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(iii). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition of the invention are further described in the C.T.F.A. Cosmetic Ingredient Dictionary and Handbook, Eleventh Edition, 2006, under the classification of “Esters”, the text of which is hereby incorporated by reference in its entirety.

### (b). Hydrocarbon Oils

It may be desirable to incorporate one or more nonvolatile hydrocarbon oils into the composition of the invention. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

### (c). Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

### (d). Nonvolatile Silicones

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

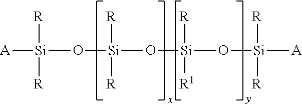

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

### G. Sunscreens

It may also be desirable to include one or more sunscreens in the compositions of the invention. Such sunscreens include chemical UVA or UVB sunscreens or physical sunscreens in the particulate form. Inclusion of sunscreens in the compositions containing the whitening active ingredient will provide additional protection to skin during daylight hours and promote the effectiveness of the whitening active ingredient on the skin. If present, the sunscreens may range from about 0.1 to 50%, preferably from about 0.5 to 40%, more preferably from about 1 to 35%.

### 1. UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds of the formula:

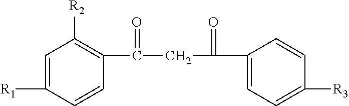

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercially available from Givaudan-Roure under the trademark Parsol® 1789, and Merck & Co. under the tradename Eusolex® 9020.

Other types of UVA sunscreens include dicamphor sulfonic acid derivatives, such as ecamsule, a sunscreen sold under the trade name Mexoryl®, which is terephthalylidene dicamphor sulfonic acid, having the formula:

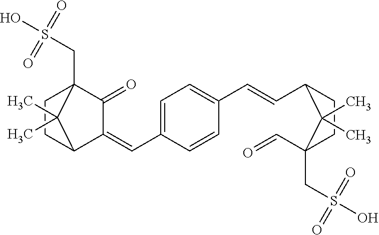 [SMILES: CC1(C)C(/C=C/c2ccc(/C=C3\C(=O)C4(CS(=O)(=O)O)CCC3C4(C)C)cc2)CCC1(C=O)CS(=O)(=O)O]

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

### 2. UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul® N-539.

Other suitable sunscreens include benzylidene camphor derivatives as set forth in U.S. Pat. No. 3,781,417, which is hereby incorporated by reference in its entirety. Such benzylidene camphor derivatives have the general formula:

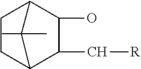

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

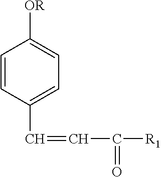

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol® MCX, or BASF under the tradename Uvinul® MC 80.

Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

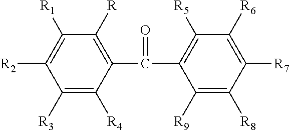

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

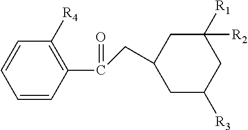

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the trademark Eusolex® HMS and menthyl anthranilate is commercially available from Haarmann & Reimer under the trademark Heliopan®. If present, the Homosalate should be found at no more than about 15% by weight of the total composition.

Various amino benzoic acid derivatives are suitable UVB absorbers including those having the general formula:

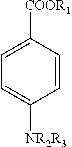

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have certain SPF (sun protective factor) values ranging from about 1-50, preferably about 2-45, most preferably about 5-30. Calculation of SPF values is well known in the art.

### H. Vitamins and Antioxidants

It may be desirable to incorporate one or more vitamins or antioxidants in the composition of the invention. If present, suggested ranges are from about 0.001 to 20%, preferably from about 0.005 to 15%, more preferably from about 0.010 to 10%. Preferably such vitamins, vitamin derivatives and/or antioxidants are operable to scavenge free radicals in the form of singlet oxygen. Such vitamins may include tocopherol or its derivatives such as tocopherol acetate, tocopherol ferulate; ascorbic acid or its derivatives such as ascorbyl palmitate, magnesium ascorbyl phosphate; Vitamin A or its derivatives such as retinyl palmitate; or vitamins D, K, B, or derivatives thereof.

While not wishing to be bound by theory, it is believed that delivery of roselle extract and betaine compound can reduce stress-induced cell damage and fight against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

Thus, the present invention is also directed to a non-therapeutic cosmetic treatment process for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin, comprising the topical application to keratin materials such as the skin, of a composition as defined above. The term “stress factor”

In one embodiment, the composition of the invention may be applied locally to the neck, the face, the neckline, the back, the hands or the body's area in an effective amount such that the combination of roselle extract and betaine compound can provide a synergistic protective property against osmotic stress.

The present invention also provides the use of roselle extract and betaine compound for the preparation of a medication for treating the cutaneous dehydration signs.

The invention will be further described in connection with the following example which is set forth for purposes of illustration only.

### EXAMPLES

### A. In Vitro Tests

### 1. Active Preparation

1.2 g betaine (RDS 51-6709) was dissolved in 100 ml water with magnetic stirring for complete dissolution (Stock concentration 1.2%). 0.5 g Roselle extract (Hibiscus Sabdariffa Extract, Lucas Meyer Cosmetics Company) was dissolved in 10 mL water to achieve 5% stock solution. Then the solutions were filtrated with 0.22 um filter to keep sterile.

### 2. Cell Culture

Normal Human Epidermal Keratinocytes (NHEK, FC-0007) purchased from Lifeline Cell Technology were incubated at 37° C., 95% humidity and 5% CO₂ in keratinocyte cell culture medium (DermaLife K Keratinocyte Medium Complete Kit, LL-0007, Lifeline cell technology) supplemented with growth factors and 1% Penicillin/Streptomycin (Thermo Fischer, Waltham, MA, USA). Cells were passaged upon reaching 80% confluency.

### 3. Cell Culture Medium Component

| | | | Final Concentrations in | |
|---|---|---|---|---|
| Product | Volume | | Supplemented Medium | |
| DermaLife K LifeFactors Kit | | | | |
| rh Insulin LifeFactor | 0.5 | mL | 5 | μg/mL |
| L-Glutamine LifeFactor | 15 | mL | 6 | mM |
| Epinephrine LifeFactor | 0.5 | mL | 1 | μM |
| Apo-Transferrin LifeFactor | 0.5 | mL | 5 | μg/mL |
| rh TGF-α LifeFactor | 0.5 | mL | 0.5 | ng/mL |
| Extract P ™ LifeFactor | 2 | mL | 0.4% | |
| Hydrocortisone Hemisuccinate | 0.5 | mL | 100 | ng/mL |
| LifeFactor | | | | |
| Antimicrobial Supplement: | | | | |

| | | | |
|---|---|---|---|
| Gentamicin and Amphotericin B | 0.5 | mL | Gentamicin 30 mg/mL |
| | | | Amphotericin B 15 μg/mL |

### 4. Hyper-Osmotic Stress Model Establishment

NHEKs were plated in 96-well plates at 4,000/well density. After 24 h of incubation, the cells would have reached ˜80% confluency. 4.38 mg/mL NaCL was added in the culture media to achieve 450 osmolarity, on top of a baseline ˜300 mOsm.

### 5. Cell Treatment

On day 1, NHEKs were seeded with culture medium (DermaLife K Keratinocyte Medium Complete Kit) in a 96-well plate at 3,000 cells/well. After 24 h adherence, a 2-active combination experiment was carried out in vitro. 21 data points were used for dose and effect, including 4 concentrations for betaine, 7 concentrations for roselle extract and their combination as below (Table 1). Cells were treated with the combination of these actives for 48 hrs (Separately diluted with cell culture medium, followed by mixing together to treat cells). Then cells were washed with Dulbecco's phosphate buffered saline (DPBS) twice, and then incubated in medium with 10% alamarBlue™ Cell Viability Reagent (Thermo Fisher, Waltham, MA, USA) for 120 minutes. The incubated plates were measured for fluorescence using a Perkin Elmer plate reader (VICTOR Nivo), at Ex 530 nm/Em 615 nm. The value are recorded as RFU value.

### 6. Cell Protection Rate

The protective effect can be determined by Cell viability ratio (%) and Cell protection rate (%) as follows.

$$Cell viability ratio ( \% ) = \frac{Experimental the combination or no treatment RFU value at 450 mOSM}{Experimental no treatment RFU value at 300 mOSM} * 100 \%$$

$$Cell protection rate ( \% ) = ( Cell viability {ratio}_{combination at 450 mOSM} - Cell viability {ratio}_{No treatment at 450 mOSM} ) / ( 100 - Cell viability {ratio}_{no treatment at 450 mOSM} ) * 100 \%$$

### 7. Combination Index (CI)

According to the theory proposed by Chou and Talalay (TING-CHAO CHOU. Theoretical Basis, Experimental Design, and Computerized Simulation of Synergism and Antagonism in Drug Combination Studies. Pharmacol Rev 58:621-681, 2006), the level of synergism can be typically measured and quantified by the drug combination index (CI, a quantitative measure of drug combination effects) from experimental dose-response data. The concentration of each compound, the protection ratio of each compound or the combo of these two compounds data were input into computer for automated analysis using CalcuSyn software. The combination index can be used to classify drug interactions as synergistic, additive. or antagonistic as below.

| | |
|---|---|
| CI < 1 | Synergy |
| CI = 1 | Additivity |
| CI > 1 | Antagonism |

### 8. Combination Indices: Effects of Betaine and Roselle Extract Concentrations, Combination Concentration Ratios and Effect Levels

Experiments were conducted at four levels of betaine and seven levels of roselle extract. Combination ratio indicates the betaine-to-roselle extract ratio in their mass concentrations. Synergy value is classified based on the calculated combination index.

TABLE 1
NHEKs treated with different concentrations of betaine and Roselle

| | | Roselle | Betaine/ | | Combi- | |
|---|---|---|---|---|---|---|
| | Betaine | extract | Roselle | Protective | nation | Synergy |
| Active | (%) | (%) | Ratio | Ratio (%) | index | or not |
| Single | 0 | | | 0 | | |
| active | 0.03 | | | 8.924 | | |
| | 0.06 | | | 19.658 | | |
| | 0.09 | | | 15.259 | | |
| | 0.1 | | | 17.451 | | |
| | | 0 | | 0 | | |
| | | 0.005 | | 3.224 | | |
| | | 0.01 | | 12.706 | | |
| | | 0.02 | | 25.234 | | |
| | | 0.05 | | 30.482 | | |
| | | 0.1 | | 24.747 | | |
| | | 0.2 | | 7.657 | | |
| Combo | 0.03 | 0.005 | 6/1 | 1.325 | >1 | No synergy |
| | 0.03 | 0.01 | 3/1 | 28.486 | <1 | Synergy |
| | 0.03 | 0.02 | 3/2 | 39.063 | <1 | Synergy |
| | 0.03 | 0.05 | 3/5 | 40.302 | <1 | Synergy |
| | 0.03 | 0.1 | 3/10 | 30.989 | <1 | Synergy |
| | 0.03 | 0.2 | 3/20 | 7.192 | >1 | No synergy |
| | 0.06 | 0.02 | 3/1 | 48.843 | <1 | Synergy |
| | 0.06 | 0.05 | 6/5 | 40.731 | <1 | Synergy |
| | 0.06 | 0.1 | 6/10 | 35.353 | <1 | Synergy |
| | 0.09 | 0.05 | 9/5 | 30.343 | <1 | Synergy |
| | 0.09 | 0.1 | 9/10 | 46.022 | <1 | Synergy |
| | 0.1 | 0.02 | 5/1 | 31.934 | <1 | Synergy |
| | 0.1 | 0.05 | 2/1 | 67.70 | <1 | Synergy |

### B. Formulation Examples

The composition of the invention containing the roselle extract and the betaine compound is prepared as follows.

| Ingredient | w/w % |
|---|---|
| Oleth-3 phosphate | 0.45 |
| Oleth-3 | 0.35 |
| Oleth-5 | 0.24 |
| Butylene glycol | 1.3 |
| Squalane | 0.5 |
| BHT | 0.1 |
| Ethylhexyl methoxycinnamate | 0.1 |
| Choleth-24/ceteth-24 | 0.1 |
| Triethanolamine | 0.61 |
| Retinyl palmitate/zea mays (corn) oil/BHT/BHA | 0.1 |
| Bisabolol | 0.1 |
| Methyl paraben | 0.46 |
| PEG-75 | 4 |
| Bis-PEG-18 methyl ether dimethyl silane | 2 |
| Glycereth-26 | 1 |
| Methyl gluceth-20 | 4 |
| Trisodium EDTA | 0.1 |
| Pantethine | 0.14 |
| Xanthan gum | 0.075 |
| Carbomer | 0.26 |
| Phenoxyethanol | 0.72 |
| Benzyl alcohol | 0.1 |
| Sodium ribonucleic acid | 0.01 |
| Sodium hyaluronate | 0.01 |
| Betaine | 0.09 |
| Roselle extract | 0.05 |
| Water | To 100 |

The composition is prepared by thoroughly mixing these ingredients in accordance with a conventional method.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. Composition, especially cosmetic and/or dermatological composition for topical application, comprising a roselle extract and at least a betaine compound selected from the group consisting of betaine, its salt and its analogs.

2. Composition according to claim 1, wherein the betaine compound is glycine betaine.

3. Composition according to claim 1, wherein the roselle extract is from the fruit of the roselle.

4. Composition according to claim 3, wherein the roselle extract is extracted by an aqueous extraction process.

5. Composition according claim 4, wherein the betaine compound and the roselle extract are present in a weight ratio from 3:1 to 3:10, preferably from 3:1 to 6:10 and more preferably from 9:5 to 9:10.

6. Composition according claim 1, wherein the betaine compound is present in an amount from 0.01% to 0.2% by weight, preferably from 0.03% to 0.1% by weight, relative to the total weight of the composition.

7. Composition according to claim 1, wherein the roselle extract is present in an amount from 0.01% to 0.1% by weight, preferably from 0.02% to 0.1% by weight and more preferably from 0.05% to 0.1% by weight, relative to the total weight of the composition.

8. Non-therapeutic cosmetic treatment process, comprising the topical application to keratin materials such as the skin of a composition as defined in claim 7.

9. Process according to claim 8 for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

10. Process according to claim 9, wherein the stress factor is UV radiation, chemical and/or osmotic stress.

11. Cosmetic use of the composition as defined in claim 7 for caring for, protecting and/or making up the skin of the body or of the face, or for caring for the hair.

12. Use according to claim 11 for fighting against the signs of a stress factor-induced osmolarity dysregulation of the epidermis, such as the appearance of fine lines and wrinkles, the withering, the thinning, the dryness, the looseness, the dull appearance, or the heterogeneous surface, of the skin.

13. Use according to claim 12, wherein the stress factor is UV radiation, chemical and/or osmotic stress.
